(19) United States
(12) Patent Application Publication
(10) Pub. No.: US 2022/0246280 A1
(43) Pub. Date: Aug. 4, 2022

(54) Methods and Systems for Assessing Brain Health Using Keyboard Data

(71) Applicant: KeyWise, Inc., Chicago, IL (US)

(72) Inventors: Raeanne C. Moore, San Diego, CA (US); Alex Leow, Chicago, IL (US); Olusola Ajilore, Chicago, IL (US); Allan Baw, Chicago, IL (US)

(21) Appl. No.: 17/455,158

(22) Filed: Nov. 16, 2021

Related U.S. Application Data
(60) Provisional application No. 63/144,793, filed on Feb. 2, 2021.

(51) Int. Cl. G16H 20/70; G16H 40/63

(52) U.S. Cl. CPC G16H 20/70; G16H 40/63

(57) ABSTRACT

An embodiment may involve receiving digital behaviorome data collected using sensors associated with a computing device and determining one or more user baseline models comprising statistical relationships between at least two of the plurality of user interaction features. The embodiment may involve receiving additional digital behaviorome data comprising a plurality of additional user interaction features corresponding to a subset of the plurality of user interaction features, selecting a particular user baseline model based on the particular user baseline model comprising statistical relationships between features of the subset of the plurality of user interaction features, determining a statistical value based on a comparison of values of the at least two of the additional user interaction features relative to the particular user baseline model and based on the statistical value being outside the predefined range, determining that the particular physical, emotional, or cognitive user characteristic for the user is within an expected range.

### CROSS-REFERENCE TO RELATED APPLICATIONS

The present application claims the benefit of U.S. Provisional Application No. 61/144,793 filed on Feb. 2, 2021, the contents of which are incorporated herein by reference in their entirety.

### BACKGROUND

Brain health may typically be assessed through clinical evaluations, diagnostic interviews, mood ratings, and other assessments that are conducted intermittently and in a controlled environment. These assessments frequently depend on a patient's self-reported symptoms and/or symptoms reported by a relevant third party (e.g., family members, caretakers, etc.), resulting in the reported symptoms being subject to recall biases, thereby making diagnoses less reliable. In addition, the reported symptoms may also often be representative of a particular period in time or sporadic, irregular periods in time and thus might not accurately illustrate the patient's symptoms and condition as a whole.

### SUMMARY

The embodiments herein present methods and accompanying systems/devices for detecting neurological and/or psychiatric disorders by collecting and analyzing digital behaviorome data for statistical relationships among user interaction features. From the collected digital behaviorome data, a baseline model may be constructed. Future collected digital behaviorome data may be compared to this baseline model to detect any physical, emotional, or cognitive user characteristic that may be within an expected range to indicate a normal and/or abnormal physical, emotional, or cognitive user characteristic.

Accordingly, a first example embodiment may involve receiving, by a processor of a computing device associated with a user, digital behaviorome data collected using sensors associated with the computing device, where the digital behaviorome data comprises a plurality of user interaction features including keystroke dynamic data representative of user keyboard usage patterns. The first example embodiment may also involve determining, by the processor, one or more user baseline models, where each of the user baseline models comprises statistical relationships between at least two of the plurality of user interaction features, where each of the user baseline models corresponds to one or more physical, emotional, or cognitive user characteristics. The first example embodiment may additionally involve receiving, by the processor, additional digital behaviorome data comprising a plurality of additional user interaction features corresponding to a subset of the plurality of user interaction features. The first example embodiment may further involve selecting, by the processor, a particular user baseline model from the one or more user baseline models based on the particular user baseline model comprising statistical relationships between features of the subset of the plurality of user interaction features, where the particular user baseline model corresponds to a particular physical, emotional, or cognitive user characteristic. The first example embodiment may further involve determining, by the processor, a statistical value based on a comparison of values of the at least two of the additional user interaction features relative to the particular user baseline model. The first example embodiment may additionally involve determining, by the processor, that the statistical value is outside a predefined range. The first example embodiment may further involve based on the statistical value being outside the predefined range, determining, by the processor, that the particular physical, emotional, or cognitive user characteristic for the user is within an expected range. The first example embodiment may also involve displaying, by the processor, that the particular physical, emotional, or cognitive user characteristic for the user is within the expected range.

In a second example embodiment, an article of manufacture may include a non-transitory computer-readable medium, having stored thereon program instructions that, upon execution by a computing system, cause the computing system to perform operations in accordance with the first example embodiment.

In a third example embodiment, a computing system may include at least one processor, as well as memory and program instructions. The program instructions may be stored in the memory, and upon execution by at least one processor, cause the computing system to perform operations in accordance with the first and/or second example embodiment.

In a fourth example embodiment, a system may include various means for carrying out each of the operations of the first, second, and/or third example embodiment.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 depicts a computing device, in accordance with example embodiments.

FIG. 2A depicts a user interface of a computing device, in accordance with example embodiments.

FIG. 2B depicts data collected by a computing device, in accordance with example embodiments.

FIG. 3A depicts a user interface of a computing device, in accordance with example embodiments.

FIG. 3B depicts data collected by a computing device, in accordance with example embodiments.

FIG. 4 depicts a user baseline model, in accordance with example embodiments.

FIG. 5 depicts a user baseline model, in accordance with example embodiments.

FIG. 6 depicts a user baseline model, in accordance with example embodiments.

FIG. 7 depicts example user interfaces, in accordance with example embodiments.

FIG. 8 depicts example user interfaces, in accordance with example embodiments.

FIG. 9 is a flow chart of a method, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein. Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

Overview

Treatment of neuropsychiatric disorders may have been hampered by the lack of objective tests of brain function relevant to such disorders. Current methods of assessing the course of these neuropsychiatric disorders and assessing the course of treatment of these neuropsychiatric disorders may be limited by biases including recency bias and recall bias. Further, these assessments may be done asynchronously, which may miss vital temporal features of symptomatic changes. These limitations may contribute to unsatisfactory clinical outcomes for patients with these disorders, as well as hampering the development of treatments for these disorders.

Provided herein are methods and accompanying systems/devices that take advantage of recent developments, e.g., the proliferation of mobile devices and the advancements in machine learning, to address these limitations. An application on a mobile device may collect digital behaviorome data on user activities using the mobile device, and the application may assess the collected digital behaviorome data for physical, emotional, and/or cognitive user functions using machine learning algorithms and statistical techniques.

In some examples, a computing device may receive digital behaviorome data collected using sensors associated with the computing device. For example, the sensors may include a touchscreen, a keyboard, one or more gyroscopes, one or more accelerometers, other sensors, and/or a combination thereof. Digital behaviorome data collected using these sensors may include a plurality of user interaction features, which may include keystroke dynamic data representative of user keyboard usage patterns. For example, the plurality of user interaction features may include individual keystrokes, transitions between individual keystrokes, pauses between keystrokes, number of pauses between keystrokes, backspace usage features, input mistakes features, input time features, typing rhythm features, accuracy features, and so on. These individual keystrokes, transitions between individual keystrokes, and pauses between keystrokes may be collected as keystroke dynamic data.

The computing device may passively collect this digital behaviorome data as the user is using the computing device during normal operations. For example, a user may be typing a message to post on social media using the computing device. The computing device may collect keystroke dynamic data and other keystroke information using the touchscreen on the computing device as the user is typing out the message to post on social media. In some examples, the computing device may also collect data using an accelerometer and/or a gyroscope on the computing device to determine whether the user set the computing device down (e.g., on a table or other surface), the angle at which the computing device is tilted while the user is typing out the note, and so on.

Based on the digital behaviorome data, the computing device may determine one or more user baseline models. Each of the user baseline models may include statistical relationships between at least two of the user interaction features and correspond to one or more physical, emotional, and/or cognitive user characteristics. In some examples, a user baseline model may include an expected distribution of inter-key delay and the frequency of the various keystroke transitions for normal and/or regular user cognitive function. In further examples, a user baseline model may include an expected clustering of points representing inattentiveness, where the cluster of points may be represented by a range of values in a high dimensional space representing the relationship between inter-key delay and frequency. And in some examples, a user baseline model may include an expected clustering of points representing the expected distribution of points as a disease progresses, where the model includes relationships between frequency, time of day, and inter-key delay. Further, in some examples, a user baseline model may be a probabilistic graphical model (e.g., a Hidden Markov Model (HMM)) that may predict a cognitive process based on the digital behaviorome data, and the cognitive process may be associated with a particular model that represents relationships between a plurality of user interaction features.

The computing device may receive additional digital behaviorome data that includes a plurality of additional user interaction features. The additional digital behaviorome data may be compared against the user baseline models described above to determine whether the user displays normal and/or abnormal physical, emotional, and/or cognitive user characteristics.

Specifically, the computing device may select a particular user baseline model from the one or more user baseline models based on the particular user baseline model including statistical relationships between the plurality of additional user interaction features with which the digital behaviorome data is associated. For example, if the additional digital behaviorome data includes inter-key delay and the frequency of each inter-key delay, the computing device may then select a user baseline model that includes relationships between inter-key delay and the frequency of each inter-key delay, e.g., a user baseline model including an expected clustering of points representing inattentiveness as described by certain relationships between inter-key delay and the frequency of each inter-key delay.

The computing device may then determine a statistical value based on a comparison of the additional user interaction features relative to the particular user baseline model. For example, the computing device may determine the likelihood that the additional inter-key delay and the additional frequency of the various keystroke transitions of the additional digital behaviorome data falls within the cluster that represents inattentiveness. For instance, if the additional inter-key delay and the additional frequency of the various keystroke transitions falls within the cluster representing inattentiveness, then the statistical value may be one. Whereas, if the additional inter-key delay and the additional frequency of the various keystroke transitions falls outside the cluster representing the inattentiveness, then the statistical value may be zero, or if the additional inter-key delay and the additional frequency of the various keystroke transitions falls on the border of the cluster representing the inattentiveness, then the statistical value may be between zero and one, depending on how close the additional inter-key delay and the additional frequency of the various keystroke transitions falls in or out of the cluster.

The computing device may determine whether the determined statistical value is outside a predefined range to determine whether the physical, emotional, or cognitive user characteristic for the user is within an expected range. For example, the determined statistical value may be a number, indicating that the additional inter-key delay and the additional frequency of the various keystroke transitions falls outside of the cluster. In some examples, the predefined range for the determined statistical value may from a first number to a second number. Thus, if the number of the determined statistical value is not within the range of the first number to the second number, then based on this determined statistical value being outside the predefined range, the computing device may determine that the physical, emotional, and/or cognitive function of the user is within the expected range (e.g., the user is not having inattentiveness).

The computing device may then display this information to the user. For example, the computing device may display on its user interface that the user is likely not being inattentive or having slowed thinking. In some examples, the computing device may also display other physical, emotional, and/or cognitive functions of the user, the trends of these physical, emotional, and/or cognitive functions (e.g., having more inattentiveness on Monday than Tuesday), recommendations to improve physical, emotional, and/or cognitive functions (e.g., more sleep, mindful breathing), a combination thereof, and/or other information/recommendations to the user.

Example Computing Devices and Cloud-Based Computing Environments

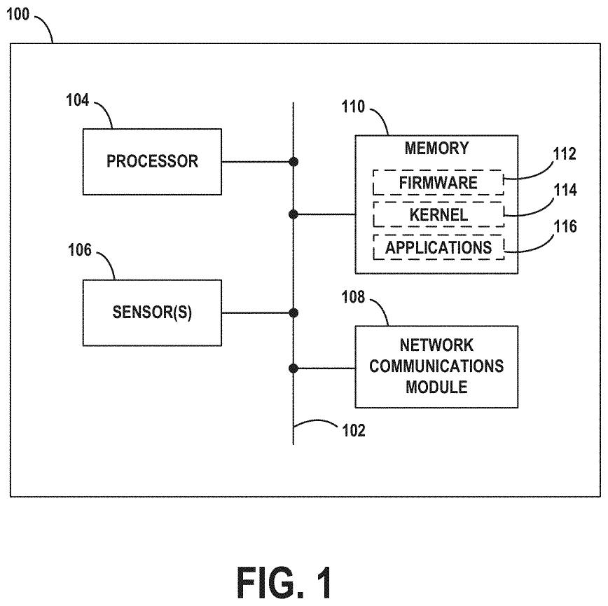

FIG. 1 is a simplified block diagram depicting example computing device 100, illustrating some of the components that may be included in a computing device arranged to operate in accordance with the embodiments herein. Computing device 100 may be a user device (e.g., a device actively operated by a user), such as a mobile device (e.g., tablet computer, smartphones, wearable computing devices) or a stationary device (e.g., desktop computers).

In this example, computing device 100 includes processor 104, one or more sensor(s) 106, network communications module 108, and memory 110, all of which may be connected by system bus 102 or a similar mechanism. In some examples, computing device 100 may include other components and/or peripheral devices (e.g., keyboards, sensors, detachable storage, printers, etc.). Additionally or alternatively, components of computing device 100 may have the ability to be decoupled. For example, sensor(s) 106 may be a detachable keyboard that connects to computing device 100.

In some examples, processor 104 may be one or more of any type of computer processing element, such as a central processing unit (CPU), a co-processor (e.g., a graphics processing unit), a network processor, and/or a form of integrated circuit or controller that performs processor operations. Processor 104 may be one or more single-core processors and/or one or more multi-core processors with multiple independent processing units. In some examples, processor 104 may include multiple types of processors. Processor 104 may also include register memory for temporarily storing instructions being executed and related data, as well as cache memory for temporarily storing recently-used instructions and data.

In some examples, sensor(s) 106 may include one or more of any type of sensor used in operations of computing device 100. For example, sensor(s) 106 may include gyroscopes, accelerometers, cameras, touchscreens, tactile buttons, keyboards, and so on. Sensor(s) 106 may be integrated onto computing device 100 (e.g., soldered onto a printed circuit board of computing device 100) or be temporarily attached onto computing device 100 (e.g., a removable keyboard or camera connected to computing device 100 via a USB peripheral). Computing device 100 may collect data from sensor(s) 106 and store them in memory 110.

Memory 110 may be any form of computer-usable memory, including but not limited to random access memory (RAM), read-only memory (ROM), and non-volatile memory (e.g., flash memory, hard disk drives, solid state drives, compact discs (CDs), digital video disks (DVDs), and/or tape storage). Thus, memory 110 may represent both temporary storage units, as well as long-term storage.

Memory 110 may store program instructions and/or data on which program instructions may operate. By way of example, memory 110 may store these program instructions on a non-transitory computer-readable medium, such that the instructions are executable by processor 104 to carry out any of the methods, processes, or operations disclosed in this specification or the accompanying drawings.

As shown in FIG. 1, memory 110 may include firmware 112, kernel 114, and/or applications 116. Firmware 112 may be program code used to boot or otherwise initiate some or all of computing device 100. Kernel 114 may be an operating system, including modules for memory management, scheduling, and management of processes, input/output, and communication. Kernel 114 may also include device drivers that allow the operating system to communicate with the hardware modules (e.g., memory units, networking interfaces, ports, and buses) of computing device 100. Applications 116 may be one or more user-space software programs, such as web browsers or email clients, as well as any software libraries used by these programs. Memory 110 may also store data used by these and other programs and applications.

Network communications module 108 may facilitate wireless communications (e.g., IEEE 802.11 (Wi-Fi), BLUETOOTH®, global positioning system (GPS), a wide-area wireless interface, or so on) and/or wired communications (e.g., Ethernet, Synchronous Optical Networking, digital subscriber line, and so on). In some examples, network communications module 108 may include one or more network communications modules and support one or more wireless and/or wired communications methods. For example, network communications module 108 may include a module that supports Wi-Fi and a module (separate or integrated) that supports BLUETOOTH®. As another example, network communications module 108 may include a module that supports Wi-Fi and a module (separate or integrated) that supports Ethernet.

Example Data Collection and Analysis

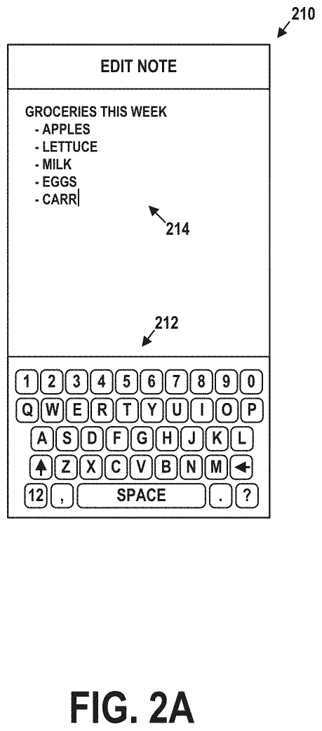

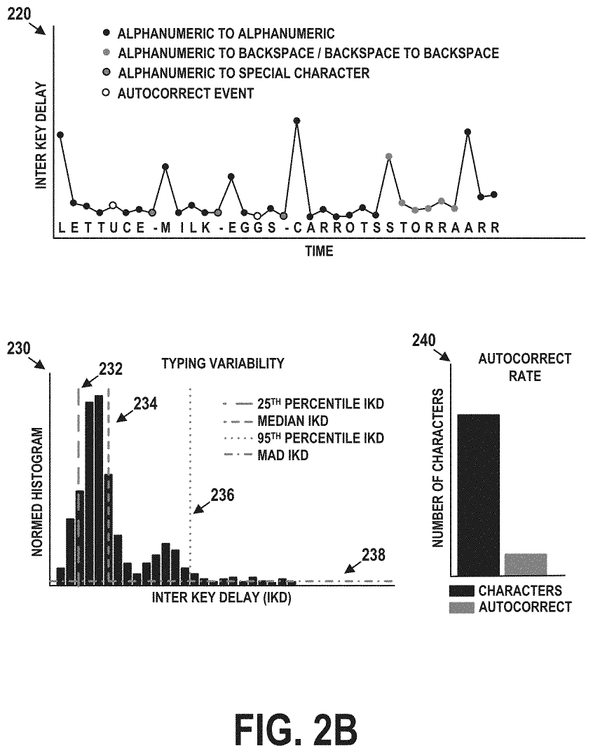

FIG. 2A depicts user interface 216 of computing device 210, in accordance with example embodiments. Computing device 210 may be an example of computing device 100 and include one or more sensors (e.g., gyroscope(s), accelerometer(s), touchscreen(s), push button(s), and so on) that may be used to collect digital behaviorome data. While a user is interacting with computing device 210, computing device 210 may passively collect digital behaviorome data using the sensors associated with computing device 210. Digital behaviorome data may include a plurality of user interaction features. For example, user interaction features may include measurements collected from gyroscopes, accelerometers, and so on. User interaction features may also include keystroke dynamic data representative of user keyboard usage patterns.

For example, computing device 210 may include a touchscreen that, at times, may display keyboard application 212, as well as other applications that are not shown. A user may use keyboard application 212 to type out text, such as note 214. While the user is using keyboard application 212, keyboard application 212 may collect and analyze keystroke dynamic data representative of user keyboard usage patterns. Specifically, keystroke dynamic data may include data collected directly from a keyboard application (e.g., keyboard application 212) and/or data collected from a keyboard application that has been analyzed. For example, keystroke dynamic data may include information such as an indication that a user pressed the letter “A” at 2:51:00 PM and/or an indication that a user pressed a first character, then a second character and the delay between pressing the two characters was 3 seconds.

In some examples, keyboard application 212 may collect timestamps of when each key on keyboard application 212 is pressed, time between each keypress, number of backspace usages, number of autocorrect occurrences, and so on as keystroke dynamic data. In some examples, keyboard application 212 may also categorize keystrokes by transition type, e.g., character-character, character-backspace, character-symbol, character-space, character-enter, alphanumeric-alphanumeric, alphanumeric-punctuation, and so on as keystroke dynamic data. This may allow for keystrokes to be categorized, while maintaining the anonymity of the actual text being entered.

In some examples, keyboard application 212 may collect the keystroke dynamic data and send the keystroke dynamic data to a database, such as a database of memory 110. In doing so, keyboard application 212 may compute and/or update statistics. For example, keyboard application 212 analyze initial keystroke dynamic data for statistics (e.g., quantile estimates, reservoir sampling, P² algorithm, and so on), and keyboard application 212 may then send and store the initial keystroke dynamic data and any determined statistics associated with the initial keystroke dynamic data. Subsequently, keyboard application 212 may receive additional keystroke dynamic data, and keyboard application 212 may update the database with the additional keystroke dynamic data and update the statistics in view of the additional keystroke data.

FIG. 2B depicts analysis that may be done using keystroke dynamic data, in accordance with example embodiments. In particular, FIG. 2B includes keystroke pattern chart 220, typing variability chart 230, and autocorrect rate chart 240. Keystroke pattern chart 220 depicts some example keystroke patterns of keystroke dynamic data that may be collected by keyboard application 212 while a user is typing note 214. Keystroke pattern chart 220 plots inter-key delay versus time. Inter-key delay may be the delay between keystrokes, and analyzing the inter-key delay may allow for insights into the user's behavior. For example, in keystroke pattern chart 220, the inter-key delay between a keystroke to hit the last letter of a line and a keystroke to hit enter may be considerably less than the inter-key delay between hitting enter and the first letter of the line. This may be expected, since a user may contemplate for a length of time before continuing to enter items into the list in notes 214.

Additionally, as mentioned above, keystroke dynamic data may include keystroke patterns that have been categorized into different transition types. For example, keystroke pattern chart 220 depicts various transition types including alphanumeric to alphanumeric, alphanumeric to backspace or backspace to backspace, alphanumeric to special character or special character to alphanumeric, alphanumeric to punctuation or punctuation to alphanumeric, autocorrect event, or a combination thereof. These transition types may be determined from the keystroke dynamic data collected by keyboard application 212 and may contribute to determining one or more cognitive and/or physical characteristics of the user.

Typing variability chart 230 depicts an example distribution of variability among inter-key delays by plotting the number of occurrences per inter-key delay length along with statistical measurements derived from the inter-key delays. Namely, typing variability chart 230 includes 25^{th} percentile inter-key delay line 232, median inter-key delay line 234, 95^{th} percentile inter-key delay line 236, and median absolute deviance inter-key delay line 238. These statistics may be obtained through analyzing keystroke dynamic data. For example, to calculate the 25^{th} percentile, keyboard application 212 may multiply 0.25 by the number of inter-key delay samples and determine, in an ordered list of inter-key delay samples, the inter-key delay sample at the resulting number. Similar calculations may be repeated for the median (50^{th} percentile inter-key delay) and the 95^{th} percentile inter-key delay. The median absolute deviance inter-key delay may be obtained through determining the median inter-key delay, calculating the deviations of each inter-key delay from the median inter-key delay value, and taking the median of those calculated deviations. Other statistics are also possible.

Autocorrect rate chart 240 depicts an example of an autocorrect rate among samples of keystroke dynamic data, represented as a bar indicating the number of characters typed and another bar indicating the number of characters that were autocorrected. Particularly, for devices with smaller keys and/or smaller keyboards, mistakes may be common and a software on the device may automatically correct for any mistakes that the user may make. For example, “lettuc” may be autocorrected to “lettuce,” “carots” may be autocorrected to “carrots,” and so on. These autocorrected letters may be counted and included in autocorrect rate chart 240 as the second column.

After keyboard application 212 collects keystroke dynamic data, keyboard application 212 may also update any statistics related to the keystroke dynamic data. For example, upon registering an additional keypress, keyboard application 212 may update keystroke pattern chart 220 with additional data points. For example, if keyboard application 212 receives an indication that the user pressed one or more additional keys, then keyboard application 212 may (1) determine the delay between the additional keypress and the previous keypress and (2) update the appropriate column in typing variability chart 230. And based on the delay between the additional keypress and the previous keypress, the calculations associated with 25^{th} percentile inter-key delay line 232, median inter-key delay line 234, 95^{th} percentile inter-key delay line 236, and median absolute deviance inter-key delay line 238 may also be updated. Further, if the additional keypress caused one or more characters, words, sentences, etc. to be autocorrected, then autocorrect rate chart 240 may also be updated. Other charts, statistics, and/or models may also be updated.

In some examples, after a length of time, a user may alter typing patterns in response to a neurological disorder, other mental disorder, a physical disorder, other disorder, or a change in behavior. As an example, the user of computing device 210 may change their behavior, e.g., a user with bipolar disorder changes from an episode of mania to an episode of depression, causing the updated keystroke dynamic data to be fairly different from the previously collected keystroke dynamic data.

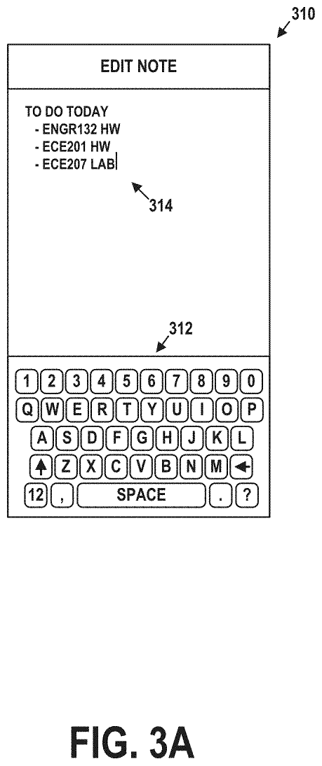

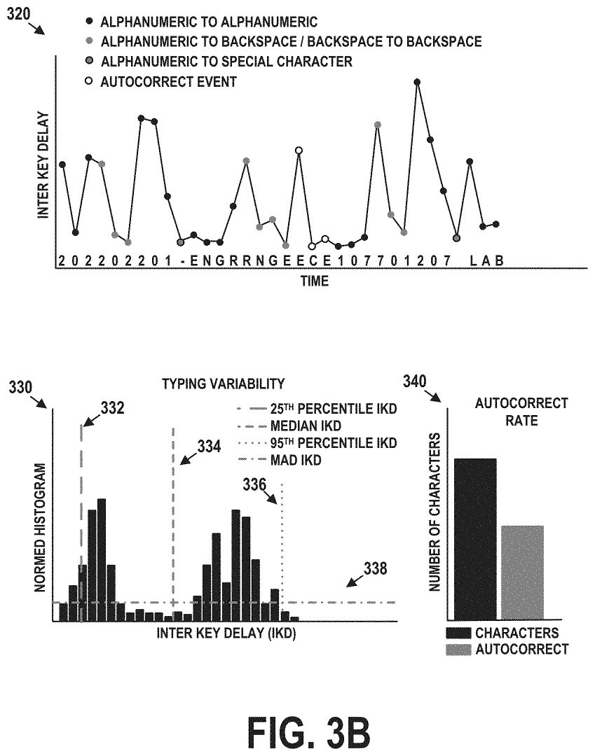

For example, FIG. 3A depicts user interface 316 of computing device 210 collecting additional digital behaviorome data, in accordance with example embodiments. The additional digital behaviorome data may be passively collected as a user of computing device 210 is using computing device 210. A user using computing device 210 may use keyboard application 212 to type out note 314, and keyboard application 212 may collect keystroke dynamic data on the user's typing patterns.

FIG. 3B depicts additional data collected by computing device 210, in accordance with example embodiments. FIG. 3B includes updated keystroke pattern chart 320, updated typing variability chart 330, and updated autocorrect rate chart. From updated keystroke pattern chart 320, it may be observed that the inter-key delays between keystrokes may be higher than before (e.g., higher than data collected a day before, for example, data depicted in FIG. 2B), the difference being more apparent in updated typing variability chart 330. Updated typing variability chart 330 may have a bimodal distribution, which may demonstrate the increase in duration of inter-key delays in comparison to typing variability chart 330. Further, updated typing variability chart 330 may include updated statistics, as indicated by updated 25^{th} percentile inter-key delay line 332, updated median inter-key delay line 334, updated 95^{th} percentile inter-key delay line 336, updated median absolute deviance inter-key delay line 338. It may be observed that the inter-key delay lines (e.g., updated median inter-key delay line 334, updated 95^{th} percentile inter-key delay line 336, updated median absolute deviance inter-key delay line 338) are all shifted to the left, which may be due to the increase in number of samples having a higher inter-key delay. Additionally, updated autocorrect rate chart 340 may indicate a higher number of samples having been updated.

Through the analysis of keystroke dynamic data as indicated by the charts in FIGS. 2B and 3B, computing device 210 may conclude that the user of computing device 210 may have changed concentration levels, become more distracted, etc. Additionally or alternatively, the changes may be indicative of a broader underlying issue, e.g., a neurological and/or physical condition.

As mentioned above, other sources of data may also be collected from the user's mobile device (e.g., computing device 210). For example, keyboard application 212 (or other application on computing device 210) may collect and store data regarding gestures registered on a touchscreen of computing device 210, user movements collected from a gyroscope and/or accelerometer of computing device 210, GPS signals from a sensor of computing device 210, and so on. Keystroke dynamic data collected by keyboard application 212 (or other application on computing device 210) as well as the other examples of data mentioned above (e.g., the data from one or more sensors on a computing device including data from gyroscopes, accelerometers, touchscreens, and so on) may be collectively referred herein as digital behaviorome data. The digital behaviorome data may be analyzed collectively to extract user behavior patterns, and be used to detect any underlying neurological and/or physical disorders.

Example Analysis Methods

To assess overall cognitive function, computing device 210 may apply unsupervised machine learning methods on the obtained digital behaviorome data. Some unsupervised machine learning methods that may be used include regression analyses, unsupervised low-dimensional embedding, latent variable inference models (e.g., HMMs), clustering methods, a combination thereof, and/or other unsupervised machine learning methods. In some examples, computing device 210 may determine one or more user baseline models, where each of the user baseline models includes statistical relationships between at least two user interaction features, and where each of the user baseline models corresponds to one or more physical, emotional, or cognitive user characteristics.

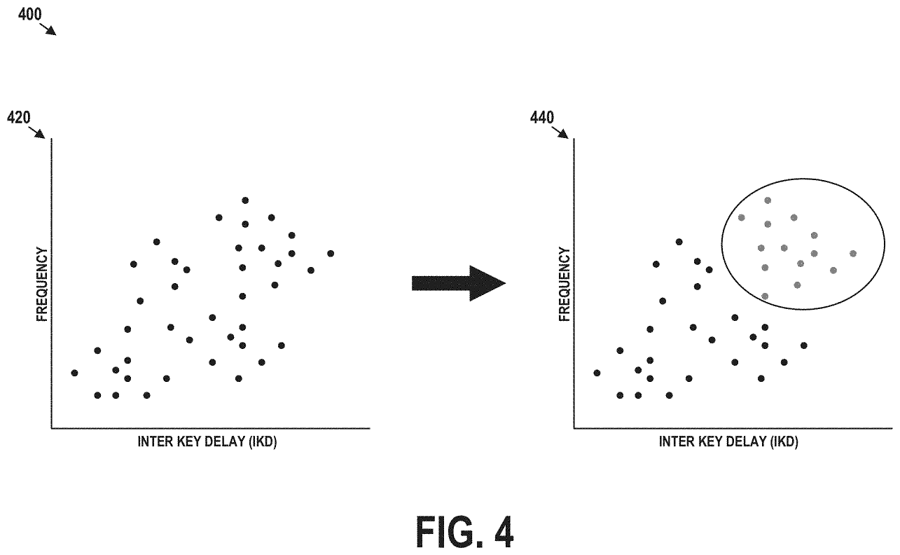

For example, FIG. 4 depicts user baseline model 400, in accordance with example embodiments. User baseline model 400 may involve clustering digital behaviorome data into different groups such that each data point of a group may correspond to whether a particular physical, emotional, or cognitive user characteristic is within an expected range. For example, user baseline model 400 includes chart 420 which may illustrate the relationship between two user interaction features, inter-key delay and frequency, for a particular user over various periods of time (e.g., frequency per day over the period of a few months). Whether the particular physical, emotional, or cognitive user characteristic is within the expected range for the user may be obtained through mathematical and/or visual analysis of chart 420. For example, user baseline model 400 also includes chart 440, which may illustrate the same relationship between inter-key delay and frequency for the same particular user over a period of time (e.g., Monday mornings), but have been analyzed (visually or mathematically) to indicate cluster 442 of abnormal data points. During the few months over which the digital behaviorome data was collected, the user may have had inattentiveness indicating a broader underlying neurological and/or physical disorder, and those inattentiveness may be indicated by cluster 442. Outside of cluster 442, digital behaviorome data that displays relatively high frequency inter-key delays with relatively long inter-key delays may be simply indicative of lack of attention that the user may have.

In some examples, the inattentiveness might not be entirely atypical (e.g., not indicative of a broader underlying neurological and/or physical disorder) unless the inattentiveness continue for a length of time. Thus, with the collection of additional digital behaviorome data, keyboard application 212 (or other application on computing device 210) may analyze the data to see where the additional digital behaviorome data falls within cluster 442. And if a significant number of points analyzed from the digital behaviorome data does fall within cluster 442, then keyboard application 212 (or other application on computing device 210) may notify the user of computing device 210 of the abnormal samples in the digital behaviorome data. In some examples, the significant number of data points indicating an abnormality may be determined through a statistical test, e.g., a Student t-test, analysis of variance (ANOVA), among many other examples.

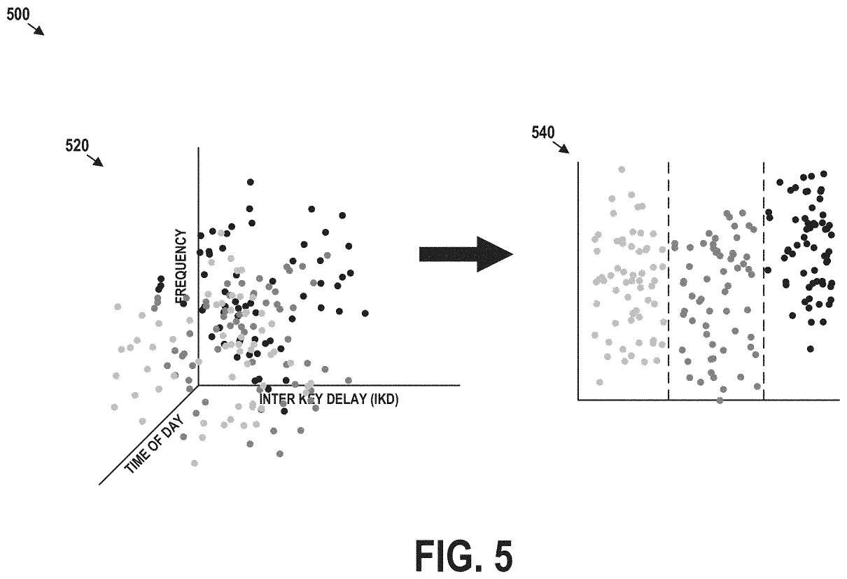

In some examples, high-dimensional and unsupervised machine learning methods may be applied to the data to reduce the dimension, before analyzing the data in the method described above. For example, FIG. 5 depicts user baseline model 500, in accordance with example embodiments. User baseline model 500 may include charts 520 and 540. Chart 520 may plot the relationship of three user interaction features, including inter-key delay, frequency, and time of day. In comparison to the digital behaviorome data plotted in charts 420 and 440 of user baseline model 400, the digital behaviorome data plotted in chart 520 may be more difficult to cluster visually due to the dimensionality of the data.

The dimensionality of the digital behaviorome data plotted in chart 520 may be reduced in a variety of ways, such as through linear methods (e.g., principal component analysis (PCA), support vector machine (SVM), and so on) and through non-linear methods (e.g., through kernelization of linear projection methods, uniform manifold approximation and projection (U-map), t-distributed scholastic neighbor embedding (t-SNE), among other non-linear methods).

Chart 540 plots the digital behaviorome data of chart 520 after the dimension has been reduced. It may be observed that, after reducing the dimensionality of the plotted keystroke dynamic data, groups of data may be clearly observed from chart 540. These groups of data may then be analyzed in a manner similar to that of charts 420 and 440 of user baseline model 400. For example, each group in chart 540 may indicate a certain property of a user that may or might not indicate an underlying neurological and/or physical disorder. For example, groups 542, 544, and 546 may indicate progressions of a disorder through time, such that keyboard usage patterns of a user is in group 542 when there is no indication of an underlying disorder, keyboard usage patterns of the user is in group 544 when there is slight indication of an underlying disorder, and keyboard usage patterns of the user is in group 546 when a disorder has progressed significantly.

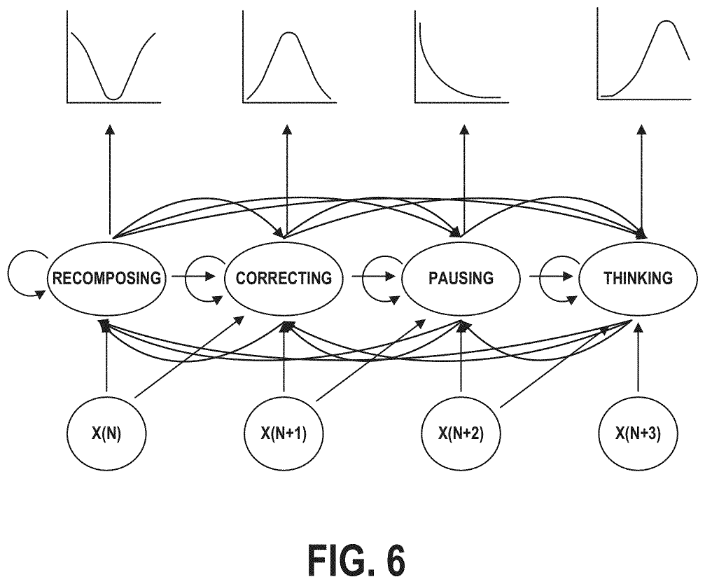

FIG. 6 depicts user baseline model 600, in accordance with example embodiments. User baseline model 600 may be used to classify digital behaviorome data in categories, such as digital behaviorome data collected when a user is composing, correcting, recomposing, pausing, waiting for others to respond, becoming distracted, thinking about what to type next, a combination thereof, among other examples. The categories may then be used to determine a baseline model (e.g., what digital behaviorome data is anticipated to resemble under normal cognitive and physical user function). Significant deviations from the baseline model may be indicative of a broader underlying disorder.

User baseline model 600 may use Hidden Markov Model (HMM) 610 to classify digital behaviorome data in categories of cognitive processes. Other probabilistic graphical models may also be used. HMM 610 may take sequential data 602 (e.g., having the same time axis) as an input. Each entry of sequential data 602 may be representative of user interaction features from sequential periods in time. For example, x(n) may be representative of the amount of inter-key delay between two keystrokes, x(n+1) may be representative of the amount of inter-key delay between two subsequent keystrokes, and so on. As another example, x(n) may be representative of statistics (e.g., 25th percentile inter-key delay, median inter-key delay, 95th percentile inter-key delay, median absolute deviance inter-key delay, autocorrect delay, or a combination thereof) collected during a period in time, x(n+1) may be representative of those user interaction features collected during a subsequent point in time, among other examples.

Keyboard application 212 may use HMM 610 to predict latent variables 604. HMM 610 may include various parameters that may represent probabilities of transitioning from a sample or samples of sequential data 602 to latent variables 604. For example, given a certain x(n), a user that caused the statistics of x(n) may have a 0.2 probability of being in the midst of recomposing a text, a 0.4 probability of being in the midst of correcting a text, 0.1 probability of being in the midst of pausing, and 0.3 probability of being in the midst of thinking about what to type. Further, HMM 610 may have transition probabilities between latent variables. For example, assuming that the user is in the midst of recomposing a text, there may be a 0.2 probability of continuing to recompose that text, 0.3 probability of correcting the text, 0.2 probability of pausing in correcting the text, and 0.3 probability of thinking about correcting the text. These various parameters of HMM 610 may be determined through variational inference.

Latent variables 604 may be representative of cognitive processes occurring during the entry of sequential data 602. Keyboard application 212 might not be able to directly observe latent variables 604, but may instead deduce the latent variables from sequential data 602. Latent variables 604 may also be sequential such that each predicted latent variable depends on the previous latent variable. For example, a user “recomposing” a message may be most likely to be “correcting” a message next, and a user “correcting” a message may be most likely to subsequently “pause” in typing a message, and so on. Other latent variables are also possible. For example, possible latent variables may further include waiting for the other person to respond, becoming distracted, among other possible cognitive processes.

Once latent variables 604 are predicted from sequential data 602, keyboard application 212 may model timing dynamics 606 associated with cognitive processes represented by latent variables 604. Timing dynamics 606 may represent distributions of inter-key delays that are associated with the cognitive processes represented by latent variables 604, as depicted by typing variability charts 230 and 330 of FIGS. 2B and 3B, respectively. For example, the cognitive process of pausing may be associated with a timing dynamic that is modeled as a power-law or a log-normal distribution that displays scale invariance across a time-scale range of naturalistic human behaviors. The parameters associated with these distributions in timing dynamics 606 may be determined through maximum likelihood estimation approximation. Computing device 210 may collect additional digital behaviorome data to be inputted into HMM 610 to determine a specific timing dynamic of timing dynamics 606, and the collected additional digital behaviorome data may be compared with the determined specific timing dynamic to determine whether a physical, emotional, or cognitive user characteristic for the user is within an expected range.

In some examples, determining one or more user baseline models may involve determining a mood stability user baseline model. To determine a mood stability user baseline model, computing device 210 may determine a variability between the plurality of user interaction features for a period of time. Based on the variability between the plurality of user interaction features, computing device 210 may also determine a threshold deviation from the variability associated with expected mood stability during the period of time, where the threshold deviation may be determined from a percentile calculation of the variability.

In some examples, the user interaction features may include a backspace usage feature, an input mistakes feature, and an input time feature. Determining one or more user baseline models may include determining an impulsivity user baseline model, which may be similar to user baseline model 500 but including relationships between a backspace usage feature, an input mistakes feature, and an input time feature. To determine the impulsivity baseline model, computing device 210 may determine a lower-dimensional projection of these features (e.g., from chart 520 to chart 540). Based on this lower-dimensional projection, computing device 210 may determine a low impulsivity time range associated with a low impulsivity user characteristic. Differences between an input time value associated with the input mistakes feature and a further input time value associated with the backspace usage feature falling within the low impulsivity time range may indicate that the user is associated with a low impulsivity user characteristic. The impulsivity user baseline model may include the low impulsivity time range, and the statistical value may be based on values of at least two of the additional user interaction features relative to the low impulsivity time range. Also based on this lower-dimensional projection, computing device 210 may determine a high impulsivity time range associated with a high impulsivity user characteristic, where the differences between an input time value associated with the input mistakes feature and a further input time associated with the backspace feature falling within the high impulsivity time range indicates that the user is associated with a high impulsivity user characteristic.

A similar method may be used to determine low and high attention ranges. In particular, the one or more user baseline models may include an attention user baseline model, and the user baseline model may include relationships between a backspace usage feature, an input mistakes feature, and an input time feature. Computing device 210 may determine a lower-dimensional projection of these three features and based on this lower-dimensional projection of these three features, computing device 210 may determine a low attention range that is associated with a low attention user characteristic. Computing device 210 may collect additional digital behaviorome data corresponding to additional user interaction features, and the statistical value may then be based on values of the additional user interaction features relative to the low attention range.

In some examples, the user baseline model may be a processing speed model that includes relationships between a typing rhythm feature and an accuracy feature. The processing speed model may be based on historical processing speed values (e.g., processing speed values for the previous week, previous month, previous year, etc.). Based on the processing speed model, computing device 210 may determine a predicted processing speed value for a period of time (e.g., a day, an hour, an afternoon, etc.) and computing device 210 may compare the predicted processing speed value for the period of time with the processing speed value of the historical processing speed values. If the processing speed value is greater than the predicted processing speed value by outside the predefined range, then computing device 210 may determine that the user processing speed characteristic for the user is within the expected range.

Other models may also be used to determine whether the one or more physical, emotional, or cognitive user characteristics falls within the expected range.

In some examples, computing device 210 may collect additional digital behaviorome data that includes a plurality of additional user interaction features, and the additional user interaction features may be used as a basis to select a particular user baseline model to use to determine whether the particular physical, emotional, or cognitive user characteristic is within an expected range. For example, computing device 210 may collect and/or determine additional user behaviorome data (e.g., the additional data depicted in FIG. 3B) including user interaction features of frequency and inter-key delay and select between the three user baseline models of FIGS. 4-6. Since FIG. 4 includes user interaction features including frequency and inter-key delay, computing device 210 may then select user baseline model 400 as the model to use to determine whether the additionally collected digital behaviorome data corresponds to inattentiveness. As another example, computing device 210 may collect and/or determine additional user behaviorome data including user interaction features of frequency, time of day, and inter-key delay. Since FIG. 4 includes these user interaction features, computing device 210 may select user baseline model 500 as the model to use to determine the progression of a disorder through time.

Additionally or alternatively, computing device 210 may select the user baseline model based on the particular physical, emotional, or cognitive user characteristic that is being determined. For example, computing device 210 may be generating graphics to show the physical, emotional, and/or cognitive health of a user of computing device 210. A particular graphic may display a user attention level, which may make use of whether the user is being inattentive, and computing device 210 may thus use user baseline model 400 as a basis to determine which user baseline model to use. Accordingly, computing device 210 may use the needed physical, emotional, or cognitive user characteristic as a basis to determine which user baseline model to use.

In some examples, after selecting a particular user baseline model to use, computing device 210 may compare the values of the additional user interaction features to the particular user baseline model. For example, if user baseline model 400 is selected, computing device 210 may analyze the additional frequency and additional inter-key delay values in the context of the clusters developed in user baseline model 400. The additional frequency and additional inter-key delay values may be plotted to determine the number of points that fall within cluster 442 as the statistical value. As another example, if user baseline model 500 is selected, computing device 210 may analyze the additional frequency values, the additional time of day values, and the additional inter-key delay values in the context of user baseline model 500. The additional frequency values, the additional time of day values, and the additional inter-key delay values may be plotted and compared with user baseline model 500 to determine a number of points that fall within a region of chart 540. As a further example, if user baseline model 600 is selected, computing device 210 may analyze the additional digital behaviorome data in the context of user baseline model 600. The additional digital behaviorome data may be inputted into HMM 610 to determine a timing distribution, and the additional digital behaviorome data may be compared with the determined timing distribution using a statistical test, e.g., a Student t-test. A Student t-test may determine a p-value that corresponds with the significance of the difference between the determined timing distribution and the additional digital behaviorome data. Other statistical tests may be used, and other statistical tests may use different measures to measure differences.

Computing device 210 may then determine whether the statistical value is outside a predefined range to indicate that the particular physical, emotional, or cognitive user characteristic associated with the particular user baseline model is within an expected range. For example, if user baseline model 400 is selected, a predefined range of three to five data points falling within the cluster may be applied. If there are more than the predefined range of data points falling within the cluster (e.g., the number of data points outside the cluster may be outside the predefined range), then the user may be being inattentive. Whereas, if there are less than the threshold value of data points falling within the cluster (e.g., the number of data points outside the cluster may be outside the predefined range), the user may also be being inattentive. As another example, if user baseline model 500 is selected, a predefined range of three to five data points falling within a particular region may indicate that the user's disease progression is at a particular stage. If they are outside the predefined range of data points within a particular region, then the user's disease progression may not be in that particular stage. Whereas, if they are inside the predefined range of data points within the particular region, then the user's disease progression may be at that particular stage. As a further example, if user baseline 600 is selected and the significance of differences between the timing distribution and the additional digital behaviorome data is quantified through a Student t-test, then the predefined range corresponding to the p-value may be from zero to 0.05. If the p-value between the timing distribution and the additional digital behaviorome data is between zero and 0.05, then the difference is significant and the user may be determined to have different recomposing, correcting, pausing, thinking, etc. If the p-value between the timing distribution and the additional digital behaviorome data is outside of the range between zero and 0.05, then the difference is not significant, and the user may be determined to have not changed their recomposing, correcting, pausing, thinking, etc. typing patterns. In each of the cases listed above, determining that the statistical value is above/outside the predefined range yields information relating to the particular physical, emotional, or cognitive user characteristic being within an expected range (e.g., a range that is indicative of a particular state of the particular physical, emotional, or cognitive user characteristic). In some examples, the predefined range may instead be a threshold value such that a statistical value above and/or below the threshold value may be indicative of a particular state of the particular physical, emotional, or cognitive characteristic.

In some examples, these classifiers may be unsupervised machine learning models that do not involve the use of a learning function with individual weights to be manipulated and optimized in order to minimize a loss function.

In some examples, digital behaviorome data comprising user interaction features may include left-right or right-left swiping of the keyboard, left-right or right-left swiping of the screen, various tapping gestures, the pressure of the input onto the user interface, the velocity and linear/angular acceleration that the user swipes or otherwise interacts with the user interface, the spatial distribution and variability of the pixels traversed during gesture inputs the spatial distribution of the optimal path of the intended texts, pauses between consecutive gesture inputs, and the transition between gesture inputs, typing, and the use of backspaces or autocorrection/autosuggestion. This digital behaviorome data may be collected by a touchscreen of computing device 210, while computing device 210 is concurrently collecting gyroscope and/or accelerometer data. In some examples, computing device 210 may also be concurrently collecting other data, including global positioning system (GPS) data, phone activity data, etc.

In some examples, linguistic features of text entered from a keyboard application, e.g., keyboard application 212, may also be included in digital behaviorome data. These linguistic features may include phonological features, morphological features, semantic features, and other features. The text may be entered as part of a chatbot conversation or within a messaging system between a user and their healthcare provider. Natural language processing algorithms (e.g., word embedding and sentiment analysis) may be applied to these features of the digital behaviorome data to passively infer cognitive domains related to language functioning. In some examples, these natural language processing algorithms may be implemented on a server device (e.g., digital behaviorome data may be sent to the server device, the server device may apply the natural language processing model. The result, e.g., the user's physical, emotional, and/or cognitive user characteristic, may be sent back to computing device 210 for display). A differential privacy algorithm may be used to further protect data security and user confidentiality.

In some examples, the processes described herein may not involve a benchmark test. For example, the user baseline model and/or the user's physical, emotional, and/or cognitive user characteristics might not be compared to a neuropsychological benchmark test such that the platform does not compare the user against a tested standard.

Example User Interfaces

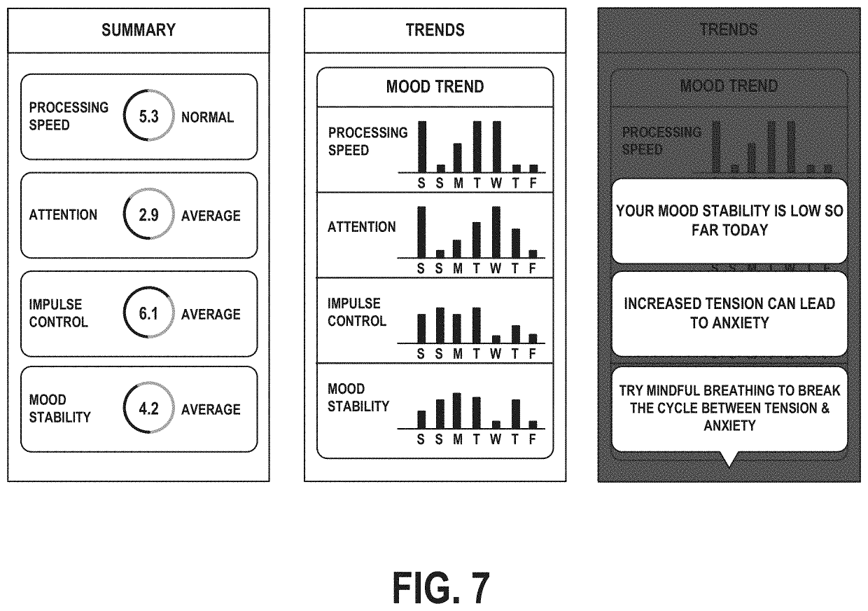

After having determined that the particular physical, emotional, or cognitive user characteristic for the user is within an expected range, computing device 210 may display that the particular physical, emotional, or cognitive user characteristic for the user is within the expected range. Additionally or alternatively, computing device 210 may display an interpretation related to the statistical value for the particular physical, emotional, or cognitive user characteristic based on the comparison of values from the at least two of the additional user interaction features relative to the particular user baseline model. FIG. 7 depicts user interface 710, user interface 720, and user interface 730, in accordance with example embodiments. Each of user interfaces 710, 720, and 730 may be used to convey various information on the determined physical, emotional, and/or cognitive user characteristics. In some examples, user interfaces 710, 720, and 730 may be displayed in real time, and these user interfaces may be presented at the momentary, daily, weekly, and monthly level with customizable alerts notifying the user (or other contact, as will be discussed later) of deviations from regular and/or average performance.

For example, user interface 710 depicts a summary of cognitive health, including metrics on processing speed, attention, impulse control, and mood stability. Each of these physical, emotional, and/or cognitive user characteristics, e.g., processing speed, attention, impulse control, and mood stability, may be determined using the methods and processes described above. In response to a change in value in a physical, emotional, and/or cognitive user characteristic, computing device 210 may update user interface 710 with the updated physical, emotional, and/or cognitive user characteristic.

In some examples, these values corresponding to the physical, emotional, and/or cognitive user characteristics may be plotted over time. For example, user interface 720 depicts processing speed, attention, impulse control, and mood stability over time. These trends may be updated periodically, e.g., every day, every few hours, etc., as new data is being collected and analyzed. If a new value corresponding to the physical, emotional, and/or cognitive user characteristic deviates significantly from what is expected and/or when the deviation occurs for an extended period of time, computing device 210 may generate and display an alert to the user that the value/trend of the physical, emotional, and/or cognitive user characteristic seems to be abnormal.

In some examples, these trends and the value of the physical, emotional, and/or cognitive user characteristic may be analyzed and improvements may be given. For example, user interface 730 depicts improvements that may be given to a user with low mood stability, including a suggestion that “increased tension can lead to anxiety” and “try mindful breathing to break the cycle between tension & anxiety.” These prompts may be customized based on what the user has seen in the prompts previously, other trends in physical, emotional, and/or cognitive user characteristics, and values of physical, emotional, and/or cognitive user characteristics. In some examples, these prompts may be implemented as tips and nudges such that computing device 210 notifies the user of these tips every now and then (e.g., every day).

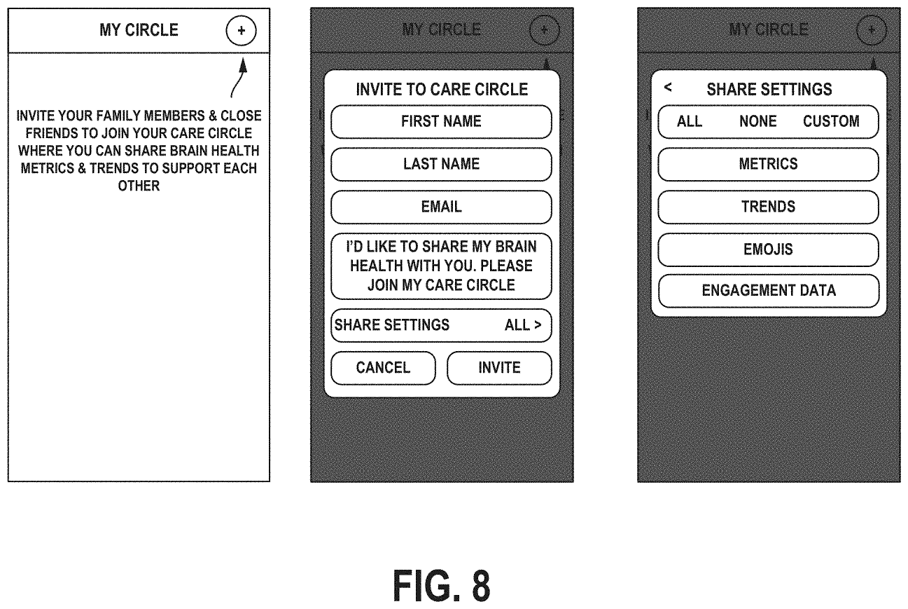

FIG. 8 depicts user interface 810, user interface 820, and user interface 830, in accordance with example embodiments. User interfaces 810, 820, and 830 depict the process of adding a contact to the user's care circle and sharing physical, emotional, and/or cognitive user characteristics with contacts in the user's care circle. For example, user interface 810 depicts inviting contacts (e.g., family members and close friends) to join the user's chare circle such that the user can share their physical, emotional, and/or cognitive user characteristics (e.g., share the values and/or trends of the physical, emotional, and/or cognitive user characteristics depicted in user interface 710 and user interface 720). User interface 820 depicts adding in a contact's information to invite them to view the user's physical, emotional, and/or cognitive user characteristics. And user interface 830 depicts a user interface where the user may set the specific information to share (e.g., all, none, custom, values, trends, emojis, engagement data, etc.). Having a care circle may be particularly useful if the user has physical, emotional, and/or cognitive disorders so that a contact may monitor them remotely. Further, a healthcare provider or researcher may be added as a contact in a care circle so that the healthcare provider or researcher may have access to the user's data and make informed treatment decisions or collect up-to-date results on how the current treatment is progressing.

Example Methods

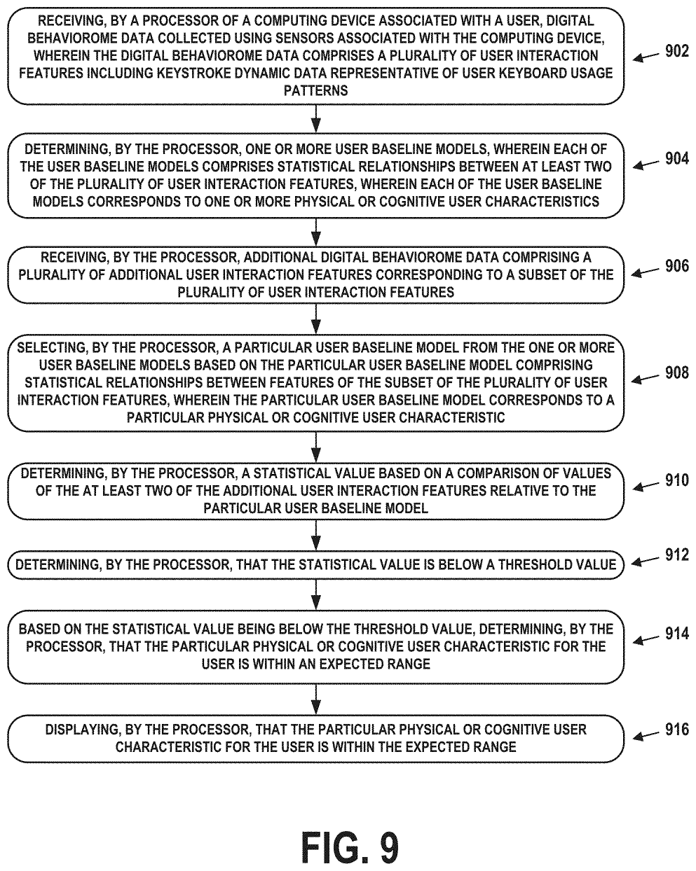

FIG. 9 is a flow chart of a method, in accordance with example embodiments. FIG. 9 may be implemented using computing device 100 and/or computing device 210. In some examples, FIG. 9 may be implemented using one or more computing devices.

At block 902, method 900 includes receiving, by a processor of a computing device associated with a user, digital behaviorome data collected using sensors associated with the computing device, where the digital behaviorome data comprises a plurality of user interaction features including keystroke dynamic data representative of user keyboard usage patterns.

At block 904, method 900 includes determining, by the processor, one or more user baseline models, where each of the user baseline models comprises statistical relationships between at least two of the plurality of user interaction features, where each of the user baseline models corresponds to one or more physical, emotional, or cognitive user characteristics.

At block 906, method 900 includes receiving, by the processor, additional digital behaviorome data comprising a plurality of additional user interaction features corresponding to a subset of the plurality of user interaction features.

At block 908, method 900 includes selecting, by the processor, a particular user baseline model from the one or more user baseline models based on the particular user baseline model comprising statistical relationships between features of the subset of the plurality of user interaction features, where the particular user baseline model corresponds to a particular physical, emotional, or cognitive user characteristic.

At block 910, method 900 includes determining, by the processor, a statistical value based on a comparison of values of the at least two of the additional user interaction features relative to the particular user baseline model.

At block 912, method 900 includes determining, by the processor, that the statistical value is outside a predefined range.

At block 914, method 900 includes based on the statistical value being outside the predefined range, determining, by the processor, that the particular physical, emotional, or cognitive user characteristic for the user is within an expected range.

At block 916, method 900 includes displaying, by the processor, that the particular physical, emotional, or cognitive user characteristic for the user is within the expected range.

In some examples, the one or more user baseline models includes a mood stability user baseline model, and determining the mood stability user baseline model of the one or more user baseline models involves: (i) determining, by the processor, variability between the plurality of user interaction features for a period of time, and (ii) based on the variability between the plurality of user interaction features, determining, by the processor, a threshold deviation from the variability associated with expected mood stability during the period of time, where the threshold deviation is determined from a percentile calculation of the variability.

In some examples, the user interaction features and the plurality of additional user interaction features each include a backspace usage feature, an input mistakes feature, and an input time feature, where the one or more user baseline models includes an impulsivity user baseline model, and where determining the impulsivity user baseline model of the one or more user baseline models involves: (i) determining, by the processor, a lower-dimensional projection of the backspace usage feature, the input mistakes feature, and the input time feature, where the lower-dimensional projection includes relationships between the backspace usage feature, the input mistakes feature, and the input time feature, and (ii) based on the lower-dimensional projection, determining a low impulsivity time range associated with a low impulsivity user characteristic, where differences between an input time value associated with the input mistakes feature and a further input time value associated with the backspace usage feature falling within the low impulsivity time range indicates that the user is associated with a low impulsivity user characteristic, where the impulsivity user baseline model includes the low impulsivity time range, and where the statistical value is based on values of at least two of the additional user interaction features relative to the low impulsivity time range.

In some examples, the user interaction features and the plurality of additional user interaction features each include a backspace usage feature, an input mistakes feature, and an input time feature, where the one or more user baseline models includes an impulsivity user baseline model, and where determining the impulsivity user baseline model of one or more user baseline models involves: (i) determining, by the processor, a lower-dimensional projection of the backspace usage feature, the input mistakes feature, and the input time feature, where the lower-dimensional projection includes relationships between the backspace usage feature, the input mistakes feature, and the input time feature, and (ii) based on the lower-dimensional projection, determining a high impulsivity time range associated with a high impulsivity user characteristic, where differences between an input time value associated with the input mistakes feature and a further input time value associated with the backspace usage feature falling within the high impulsivity time range indicates that the user is associated with a high impulsivity user characteristic, where the impulsivity user baseline model includes the high impulsivity time range and where the statistical value is based on values of the at least two of the additional user interaction features relative to the high impulsivity time range.

In some examples, the user interaction features and the plurality of additional user interaction features each include a backspace usage feature, an input mistakes feature, and an input time feature, where the one or more user baseline models includes an attention user baseline model, and where determining the attention user baseline model of one or more user baseline models involves: (i) determining, by the processor, a lower-dimensional projection of the backspace usage feature, the input mistakes feature, and the input time feature, where the lower-dimensional projection includes relationships between the backspace usage feature, the input mistakes feature, and the input time feature, and (ii) based on the lower-dimensional projection, determining a low attention range representing a range of high numbers of mistakes per time period, where the mistakes are associated with the input mistakes feature and the time period is associated with the input time feature, where the low attention range is associated with a low attention user characteristic, where the attention user baseline model includes the low attention range, and where the statistical value is based on values of the at least two of the additional user interaction features relative to the low attention range.

In some examples, the user interaction features and the plurality of additional user interaction features each include a backspace usage feature, an input mistakes feature, and an input time feature, where the one or more user baseline models includes an attention user baseline model, and where determining the attention user baseline model of the one or more user baseline models involves: (i) determining, by the processor, a lower-dimensional projection of the backspace usage feature, the input mistakes feature, and the input time feature, where the lower-dimensional projection includes relationships between the backspace usage feature, the input mistakes feature, and the input time feature, and (ii) based on the lower-dimensional projection, determining a high attention range representing a range of low numbers of mistakes per time period, where the mistakes are associated with the input mistakes feature and the time period is associated with the input time feature, where the high attention range is associated with a high attention user characteristic, where the attention user baseline model includes the high attention range, and where the statistical value is based on values of at least two of the additional user interaction features relative to the high attention range.

In some examples, method 900 further includes classifying the keystroke dynamic data representative of the user keyboard usage patterns into a plurality of keypress transition categories including character-character entry, character-backspace entry, character-space entry, character-number entry, and special character-character entry, where determining the user baseline models is further based on the classified keystroke dynamic data.

In some examples, method 900 further includes based on the digital behaviorome data, updating the one or more user baseline models.

In some examples, the user interaction features and the plurality of additional user interaction features each include a typing rhythm feature, an accuracy feature, where the one or more user baseline models includes a processing speed model, where the particular physical, emotional, or cognitive user characteristic that the particular user baseline model corresponds to is a user processing speed characteristic, and where determining the processing speed model of the one or more user baseline models involves: (i) determining, by the processor, the processing speed model based on a plurality of historical processing speed values, (ii) determining, by the processor and based on the processing speed model, a predicted processing speed value for a period of time, (iii) determining, by the processor, a processing speed value for the period of time based on the processing speed value for the period of time being higher when value of the typing rhythm feature and the accuracy feature for the period of time are higher and based on the processing speed value for the period of time, and (iv) based on the processing speed value being greater than the predicted processing speed value by less than a threshold value, determining that the user processing speed characteristic for the user is within the expected range.

In some examples, displaying that the particular physical, emotional, or cognitive user characteristic for the user is within the expected range involves: (i) determining, based on the particular user baseline model, an expected physical, emotional, or cognitive user characteristic, and (ii) displaying the particular physical, emotional, or cognitive user characteristic relative to the expected physical, emotional, or cognitive user characteristic.

In some examples, displaying that the particular physical, emotional, or cognitive user characteristic for the user is within the expected range involves: (i) displaying, by the processor, a graphic representing historical values of the particular physical, emotional, or cognitive user characteristic associated with the user.

In some examples, method 900 further includes displaying an interpretation related to the statistical value for the particular physical, emotional, or cognitive user characteristic determined based on the comparison of values from at least two of the additional user interaction features relative to the particular user baseline model.

In some examples, the digital behaviorome data is stored in a database of the computing device, and where the stored digital behaviorome data excludes user-identifying information.

In some examples, the digital behaviorome data is stored in a remote server, where the digital behaviorome data excludes user-identifying information, where the remote server also stores additional digital behaviorome data associated with a plurality of additional users, and where the particular user baseline model is based on the additional digital behaviorome data associated with the plurality of additional users.

In some examples, the sensors comprise a physical keyboard and/or a user display capable of receiving user input, where the keystroke dynamic data is collected using the physical keyboard and/or a keyboard displayed on the user display of the computing device.

In some examples, the computing device is a mobile computing device.

In some examples, the sensors comprise an accelerometer, a gyroscope, or both the accelerometer and the gyroscope, and where the digital behaviorome data is partially or entirely collected from the accelerometer, the gyroscope, or both the accelerometer and the gyroscope.

### CONCLUSION

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer readable medium such as a storage device including RAM, a disk drive, a solid state drive, or another storage medium.

The computer readable medium can also include non-transitory computer readable media such as computer readable media that store data for short periods of time like register memory and processor cache. The computer readable media can further include non-transitory computer readable media that store program code and/or data for longer periods of time. Thus, the computer readable media may include secondary or persistent long-term storage, like ROM, optical or magnetic disks, solid state drives, or compact-disc read only memory (CD-ROM), for example. The computer readable media can also be any other volatile or non-volatile storage systems. A computer readable medium can be considered a computer readable storage medium, for example, or a tangible storage device.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A method comprising:
receiving, by a processor of a computing device associated with a user, digital behaviorome data collected using sensors associated with the computing device, wherein the digital behaviorome data comprises a plurality of user interaction features including keystroke dynamic data representative of user keyboard usage patterns;
determining, by the processor, one or more user baseline models, wherein each of the user baseline models comprises statistical relationships between at least two of the plurality of user interaction features, wherein each of the user baseline models corresponds to one or more physical, emotional, or cognitive user characteristics;
receiving, by the processor, additional digital behaviorome data comprising a plurality of additional user interaction features corresponding to a subset of the plurality of user interaction features;
selecting, by the processor, a particular user baseline model from the one or more user baseline models based on the particular user baseline model comprising statistical relationships between features of the subset of the plurality of user interaction features, wherein the particular user baseline model corresponds to a particular physical, emotional, or cognitive user characteristic;
determining, by the processor, a statistical value based on a comparison of values of the at least two of the additional user interaction features relative to the particular user baseline model;
determining, by the processor, that the statistical value is outside a predefined range;
based on the statistical value being outside the predefined range, determining, by the processor, that the particular physical, emotional, or cognitive user characteristic for the user is within an expected range; and
displaying, by the processor, that the particular physical, emotional, or cognitive user characteristic for the user is within the expected range.

2. The method of claim 1, wherein the one or more user baseline models includes a mood stability user baseline model, and wherein determining the mood stability user baseline model of the one or more user baseline models comprises:
determining, by the processor, variability between the plurality of user interaction features for a period of time; and
based on the variability between the plurality of user interaction features, determining, by the processor, a threshold deviation from the variability associated with expected mood stability during the period of time, wherein the threshold deviation is determined from a percentile calculation of the variability.

3. The method of claim 1, wherein the user interaction features and the plurality of additional user interaction features each include a backspace usage feature, an input mistakes feature, and an input time feature, wherein the one or more user baseline models includes an impulsivity user baseline model, and wherein determining the impulsivity user baseline model of the one or more user baseline models comprises:
determining, by the processor, a lower-dimensional projection of the backspace usage feature, the input mistakes feature, and the input time feature, wherein the lower-dimensional projection includes relationships between the backspace usage feature, the input mistakes feature, and the input time feature; and
based on the lower-dimensional projection, determining a low impulsivity time range associated with a low impulsivity user characteristic, wherein differences between an input time value associated with the input mistakes feature and a further input time value associated with the backspace usage feature falling within the low impulsivity time range indicates that the user is associated with a low impulsivity user characteristic, wherein the impulsivity user baseline model includes the low impulsivity time range, and wherein the statistical value is based on values of at least two of the additional user interaction features relative to the low impulsivity time range.

4. The method of claim 1, wherein the user interaction features and the plurality of additional user interaction features each include a backspace usage feature, an input mistakes feature, and an input time feature, wherein the one or more user baseline models includes an impulsivity user baseline model, and wherein determining the impulsivity user baseline model of one or more user baseline models comprises:
determining, by the processor, a lower-dimensional projection of the backspace usage feature, the input mistakes feature, and the input time feature, wherein the lower-dimensional projection includes relationships between the backspace usage feature, the input mistakes feature, and the input time feature; and
based on the lower-dimensional projection, determining a high impulsivity time range associated with a high impulsivity user characteristic, wherein differences between an input time value associated with the input mistakes feature and a further input time value associated with the backspace usage feature falling within the high impulsivity time range indicates that the user is associated with a high impulsivity user characteristic, wherein the impulsivity user baseline model includes the high impulsivity time range and wherein the statistical value is based on values of the at least two of the additional user interaction features relative to the high impulsivity time range.

5. The method of claim 1, wherein the user interaction features and the plurality of additional user interaction features each include a backspace usage feature, an input mistakes feature, and an input time feature, wherein the one or more user baseline models includes an attention user baseline model, and wherein determining the attention user baseline model of the one or more user baseline models comprises:
determining, by the processor, a lower-dimensional projection of the backspace usage feature, the input mistakes feature, and the input time feature, wherein the lower-dimensional projection includes relationships between the backspace usage feature, the input mistakes feature, and the input time feature; and
based on the lower-dimensional projection, determining a low attention range representing a range of high numbers of mistakes per time period, wherein the mistakes are associated with the input mistakes feature and the time period is associated with the input time feature, wherein the low attention range is associated with a low attention user characteristic, wherein the attention user baseline model includes the low attention range, and wherein the statistical value is based on values of the at least two of the additional user interaction features relative to the low attention range.

6. The method of claim 1, wherein the user interaction features and the plurality of additional user interaction features each include a backspace usage feature, an input mistakes feature, and an input time feature, wherein the one or more user baseline models includes an attention user baseline model, and wherein determining the attention user baseline model of the one or more user baseline models comprises:
determining, by the processor, a lower-dimensional projection of the backspace usage feature, the input mistakes feature, and the input time feature, wherein the lower-dimensional projection includes relationships between the backspace usage feature, the input mistakes feature, and the input time feature; and
based on the lower-dimensional projection, determining a high attention range representing a range of low numbers of mistakes per time period, wherein the mistakes are associated with the input mistakes feature and the time period is associated with the input time feature, wherein the high attention range is associated with a high attention user characteristic, wherein the attention user baseline model includes the high attention range, and wherein the statistical value is based on values of the at least two of the additional user interaction features relative to the high attention range.

7. The method of claim 1, further comprising:
classifying the keystroke dynamic data representative of the user keyboard usage patterns into a plurality of keypress transition categories including character-character entry, character-backspace entry, character-space entry, character-number entry, and special character-character entry, wherein determining the user baseline models is further based on the classified keystroke dynamic data.

8. The method of claim 1, further comprising:
based on the digital behaviorome data, updating, the one or more user baseline models.

9. The method of claim 1, wherein the user interaction features and the plurality of additional user interaction features each include a typing rhythm feature, an accuracy feature, wherein the one or more user baseline models includes a processing speed model, wherein the particular physical, emotional, or cognitive user characteristic that the particular user baseline model corresponds to is a user processing speed characteristic, and wherein determining the processing speed model of the one or more user baseline models comprises:
determining, by the processor, the processing speed model based on a plurality of historical processing speed values;
determining, by the processor and based on the processing speed model, a predicted processing speed value for a period of time;
determining, by the processor, a processing speed value for the period of time based on the processing speed value for the period of time being higher when value of the typing rhythm feature and the accuracy feature for the period of time are higher and based on the processing speed value for the period of time; and
based on the processing speed value being greater than the predicted processing speed value by less than a threshold value, determining that the user processing speed characteristic for the user is within the expected range.

10. The method of claim 1, wherein displaying that the particular physical, emotional, or cognitive user characteristic for the user is within the expected range comprises:
determining, based on the particular user baseline model, an expected physical, emotional, or cognitive user characteristic; and
displaying the particular physical, emotional, or cognitive user characteristic relative to the expected physical, emotional, or cognitive user characteristic.

11. The method of claim 1, wherein displaying that the particular physical, emotional, or cognitive user characteristic for the user is within the expected range comprises:
displaying, by the processor, a graphic representing historical values of the particular physical, emotional, or cognitive user characteristic associated with the user.

12. The method of claim 1, further comprising:
displaying an interpretation related to the statistical value for the particular physical, emotional, or cognitive user characteristic determined based on the comparison of values from the at least two of the additional user interaction features relative to the particular user baseline model.

13. The method of claim 1, wherein the digital behaviorome data is stored in a database of the computing device, and wherein the stored digital behaviorome data excludes user-identifying information.

14. The method of claim 1, wherein the digital behaviorome data is stored in a remote server, wherein the digital behaviorome data excludes user-identifying information, wherein the remote server also stores additional digital behaviorome data associated with a plurality of additional users, and wherein the particular user baseline model is based on the additional digital behaviorome data associated with the plurality of additional users.

15. The method of claim 1, wherein the sensors comprise a physical keyboard and/or a user display capable of receiving user input, wherein the keystroke dynamic data is collected using the physical keyboard and/or a keyboard displayed on the user display of the computing device.

16. The method of claim 1, wherein the computing device is a mobile computing device.

17. The method of claim 1, wherein the sensors comprise an accelerometer, a gyroscope, or both the accelerometer and the gyroscope, and wherein the digital behaviorome data is partially or entirely collected from the accelerometer, the gyroscope, or both the accelerometer and the gyroscope.

18. A computing device comprising:
a processor; and
a non-transitory computer-readable storage medium, having stored thereon program instructions that, upon execution by the processor, cause performance of a set of operations, comprising:
receiving, by the processor of a computing device associated with a user, digital behaviorome data collected using sensors associated with the computing device, wherein the digital behaviorome data comprises a plurality of user interaction features including keystroke dynamic data representative of user keyboard usage patterns;
determining, by the processor, one or more user baseline models, wherein each of the user baseline models comprises statistical relationships between at least two of the plurality of user interaction features, wherein each of the user baseline models corresponds to one or more physical, emotional, or cognitive user characteristics;
receiving, by the processor, additional digital behaviorome data comprising a plurality of additional user interaction features corresponding to a subset of the plurality of user interaction features;
selecting, by the processor, a particular user baseline model from the one or more user baseline models based on the particular user baseline model comprising statistical relationships between features of the subset of the plurality of user interaction features, wherein the particular user baseline model corresponds to a particular physical, emotional, or cognitive user characteristic;
determining, by the processor, a statistical value based on a comparison of values of the at least two of the additional user interaction features relative to the particular user baseline model;
determining, by the processor, that the statistical value is outside a predefined range;
based on the statistical value being outside the predefined range, determining, by the processor, that the particular physical, emotional, or cognitive user characteristic for the user is within an expected range; and
displaying, by the processor, that the particular physical, emotional, or cognitive user characteristic for the user is within the expected range.

19. The computing device of claim 18, wherein the one or more user baseline models includes a mood stability user baseline model, and wherein determining the mood stability user baseline model of the one or more user baseline models comprises:
determining, by the processor, variability between the plurality of user interaction features for a period of time; and
based on the variability between the plurality of user interaction features, determining, by the processor, a threshold deviation from the variability associated with expected mood stability during the period of time, wherein the threshold deviation is determined from a percentile calculation of the variability.

20. A non-transitory computer readable medium comprising program instructions executable by at least one processor to cause the at least one processor to perform functions comprising:
receiving, by a processor of a computing device associated with a user, digital behaviorome data collected using sensors associated with the computing device, wherein the digital behaviorome data comprises a plurality of user interaction features including keystroke dynamic data representative of user keyboard usage patterns;
determining, by the processor, one or more user baseline models, wherein each of the user baseline models comprises statistical relationships between at least two of the plurality of user interaction features, wherein each of the user baseline models corresponds to one or more physical, emotional, or cognitive user characteristics;
receiving, by the processor, additional digital behaviorome data comprising a plurality of additional user interaction features corresponding to a subset of the plurality of user interaction features;
selecting, by the processor, a particular user baseline model from the one or more user baseline models based on the particular user baseline model comprising statistical relationships between features of the subset of the plurality of user interaction features, wherein the particular user baseline model corresponds to a particular physical, emotional, or cognitive user characteristic;
determining, by the processor, a statistical value based on a comparison of values of the at least two of the additional user interaction features relative to the particular user baseline model;
determining, by the processor, that the statistical value is outside a predefined range;
based on the statistical value being outside the predefined range, determining, by the processor, that the particular physical, emotional, or cognitive user characteristic for the user is within an expected range; and
displaying, by the processor, that the particular physical, emotional, or cognitive user characteristic for the user is within the expected range.
